(19) United States
(12) Patent Application Publication
(10) Pub. No.: US 2012/0330179 A1
(43) Pub. Date: Dec. 27, 2012

(54) ELECTRODE CONTACT-QUALITY EVALUATION

(75) Inventors: Jong-Tae Yuk, Redmond, WA (US); Gerald McMorrow, Redmond, WA (US)

(73) Assignee: VERATHON, INC., Bothell, WA (US)

(21) Appl. No.: 13/168,841

(22) Filed: Jun. 24, 2011

(51) Int. Cl. A61B 5/04

(52) U.S. Cl. 600/547

(57) ABSTRACT

A system includes an array of N electrode elements configured to be attached to an external region of a patient, and a processing device coupled to the array. The processing device is configured to receive a set of bioelectric data signals from the array, determine from the set of data signals a set of elements of the array that are, according to a predetermined standard, insufficiently attached to the external region, and generate to a display device, in at least two dimensions, a representation of the external region and the spatial positioning of the insufficiently attached set of elements on the external region.

### BACKGROUND OF THE INVENTION

Sensor devices that can monitor bioelectric data from a body are known. An example of such a device is described in U.S. Pat. No. 6,055,448. The apparatus described therein comprises an array of a plurality of N number of sensors where N is an integer, each sensor of which is capable of detecting an electrical signal associated with components of a heartbeat. In an associated known approach to monitoring the electrical signals detected by the sensors, executable instructions implemented by a processing device generate an indication of the quality of contact between each respective sensor of the array and the body of a patient, the heartbeat of whom the sensors are to monitor. Poor quality of contact between a sensor of the array and the body of the patient will produce a poor-quality signal, thereby preventing an optimal evaluation of the monitored heartbeat.

While this quality-of-contact evaluation functions to specifically indicate which one(s) of the sensor(s) is in poor contact with the patient, in the instance in which N is a comparatively high number, it is nonetheless difficult for a practitioner employing the sensor array to discern the specific position on the patient's body at which sensor contact quality is poor or otherwise insufficient.

### SUMMARY OF THE INVENTION

In an embodiment, a system includes an array of N electrode elements configured to be attached to an external region of a patient, and a processing device coupled to the array. The processing device is configured to receive a set of bioelectric data signals from the array, determine from the set of data signals a set of elements of the array that are, according to a predetermined standard, insufficiently attached to the external region, and generate to a display device, in at least two dimensions, a representation of the external region and the spatial positioning of the insufficiently attached set of elements on the external region.

### BRIEF DESCRIPTION OF THE DRAWING

Preferred and alternative embodiments of the present invention are described in detail below with reference to the following figures:

FIG. 1 is a high-level block diagram showing an ECG system 100 in accordance with an embodiment of the invention;

FIG. 2 is a schematic illustration of an arrangement of N electrodes in an array in accordance with an embodiment of the invention

FIG. 3 illustrates a graphical user interface according to an embodiment of the invention; and

FIG. 4 illustrates an exemplary respective connector-pin assignment for interfaces of the connector element 120 and console 130 of FIG. 1.

### DETAILED DESCRIPTION OF THE PREFERRED EMBODIMENT

Embodiments of the invention are operational with numerous general purpose or special purpose computing system environments or configurations. Examples of well known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

Embodiments of the invention may be described in the general context of computer-executable instructions, such as program modules, being executed by a computer and/or by computer-readable media on which such instructions or modules can be stored. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

Embodiments of the invention may include or be implemented in a variety of computer readable media. Computer readable media can be any available media that can be accessed by a computer and includes both volatile and nonvolatile media, removable and non-removable media. By way of example, and not limitation, computer readable media may comprise computer storage media and communication media. Computer storage media include volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

An embodiment of the invention enables a display device to display on a 3-D model of a torso the current status of electrode connectivity to assist a user in correcting poor-quality electrode contacts, as appropriate.

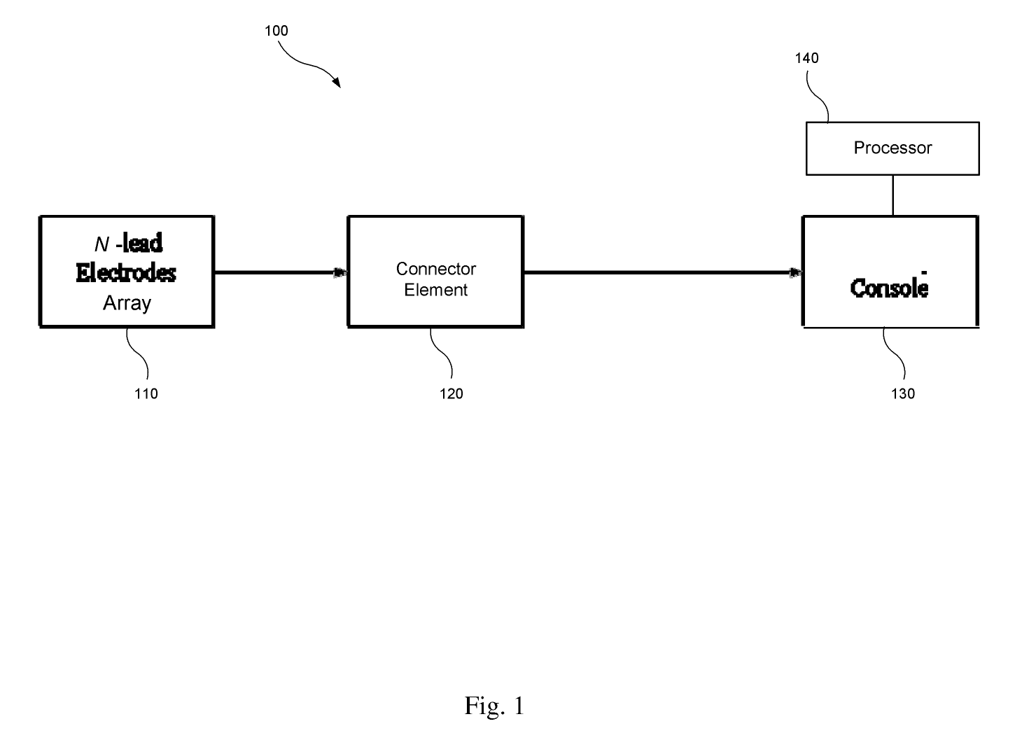

FIG. 1 is a high-level block diagram showing an ECG system 100 according to an embodiment. System 100 includes an N-lead electrode array 110, a connector element 120, and a signal-monitoring console 130 including, or otherwise coupled to, a processing device (processor) 140.

According to an embodiment of the invention, the processor 140 employs a chip (not shown), such as a Texas Instruments® AD 1298 chip, 8 channel 24 bit ECG AFE, for ECG data acquisition. Alternatively, the chip may be a component of the connector element 120. This chip provides a “lead-off” detection function using, for example, internal 10 MΩ pull-up resistors to detect whether one or more electrodes of the array 110 is in poor contact with the body of a patient (not shown). The indication of “lead-off” may be binary (i.e., ON or OFF).

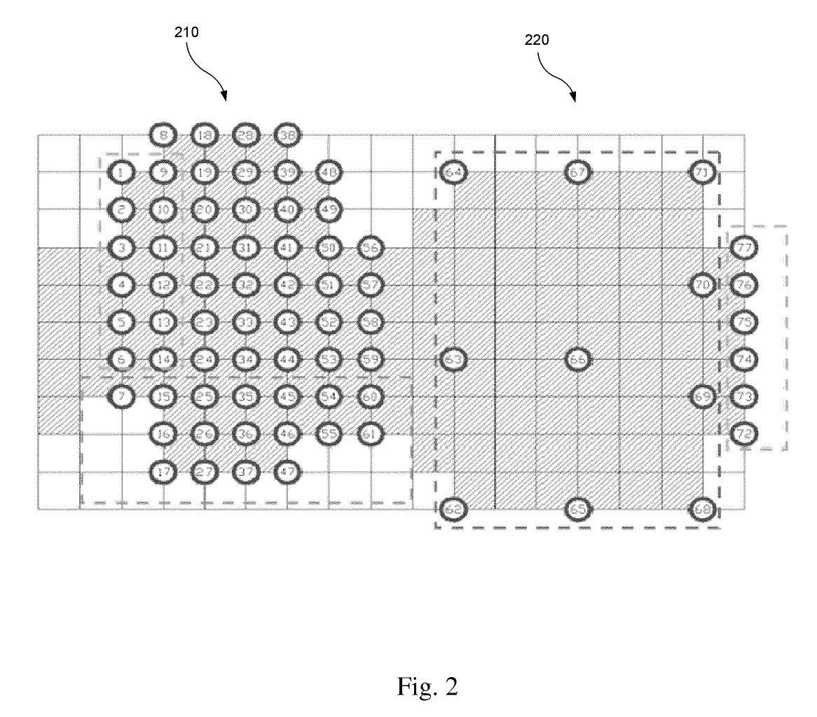

FIG. 2 is a schematic illustration of the arrangement of the N electrodes in an embodiment of the array 110. In the illustrated embodiment, the array 110 includes an anterior sub-array 210 (i.e., leads 1-61) configured to be positioned on the front of a patient's torso and a posterior sub-array 220 (i.e., leads 62-77) configured to be positioned on the back of a patient's torso. The connector element 120 may be configured to provide a common electrical interface to the console 130 for both the anterior and posterior sub-arrays 210, 220.

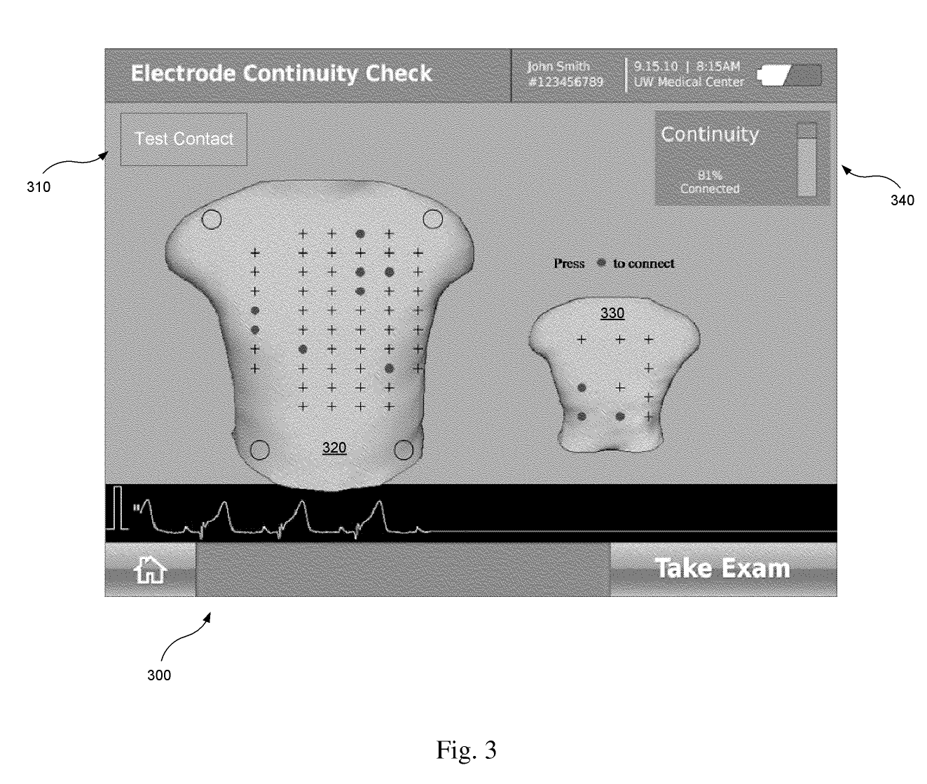

FIG. 3 illustrates a graphical user interface 300 according to an embodiment that may be employed by a user of the system 100 to perform a lead-contact-quality check. Once each lead of the array 110 has been attached to the patient, the user may, using a conventional pointer device, select a test-initiation button 310 to commence the contact-quality check.

Upon completion of the check, the interface 300 may display a first representation 320 of the front of the patient torso and the spatial positioning of sufficiently and insufficiently attached leads of the anterior sub-array 210. The interface 300 may additionally display a second representation 330 of the back of the patient torso and the spatial positioning of sufficiently and insufficiently attached leads of the posterior sub-array 220. The sufficiently attached leads may be illustrated in the interface 300 in a first format (e.g., “+” signs, as shown in FIG. 3) different from a second format (e.g., dots, as shown in FIG. 3) in which the insufficiently attached leads are illustrated. In this manner, the system 100 offers the user a more-intuitive “mapping” of the torso location of leads that require corrective attachment.

Each of the representations 320, 330 may be rotated in three dimensions within the interface 300, using a conventional input device, by the user to offer multiple views of the positioning of insufficiently attached leads relative to the patient's torso. Additionally, the interface 300 may include an indication, such as a meter 340, of the quantity of the insufficiently attached leads.

It may be desirable to ensure compatibility between the connector element 120 and console 130 as a means of enabling, or disabling, electrical communication between the console and the array 110. In an embodiment, this may be achieved by employing pull-up and/or pull-down combinations in the connector pins of the connector element 120 and console 130 as a means of implementing an “identification code.”

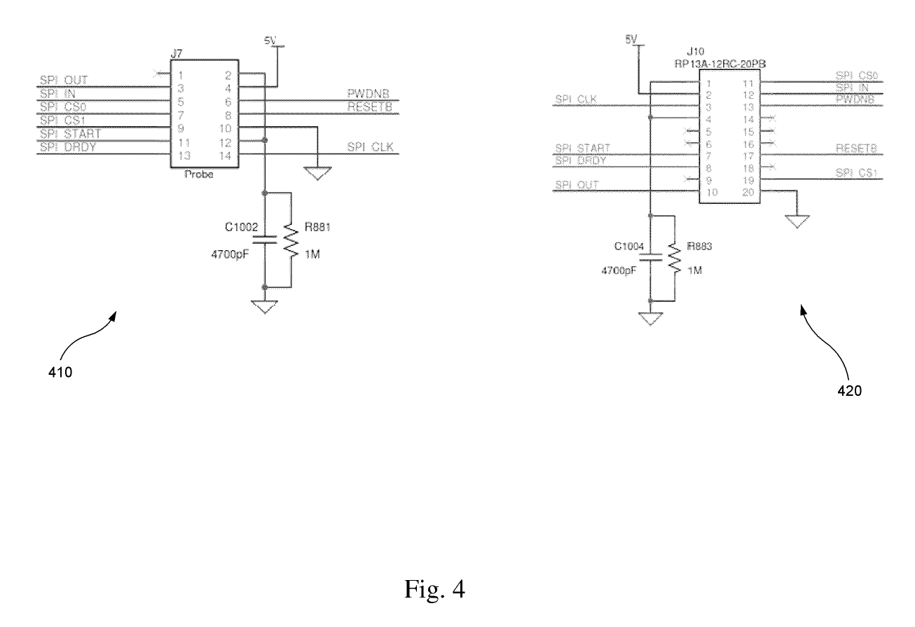

In such an embodiment, the array 110 is connected by pin connection to the connector element 120. In turn, the connector element 120 may be connected to the console 130 with, for example, 20-wire cable. An exemplary respective connector-pin assignment for an interface 410 of the connector element 120 and an interface 420 of the console 130 is illustrated in FIG. 4.

Table 1 illustrates an exemplary pin assignment table describing the connection between interfaces 410 and 420.

TABLE 1

| | Interface | Interface | |
|---|---|---|---|
| Name | 420 | 410 | Status |
| GND (high imp) | 1 | 2 | |
| 5 V | 2 | 4 | |
| SPI_CLK | 3 | 14 | |
| GND (high imp) | 4 | 12 | |
| | 5 | | |
| | 6 | | |
| SPI_START | 7 | 11 | |
| SPI_DRDY | 8 | 13 | |
| | 9 | 1 | 0 |
| SPI_OUT | 10 | 3 | |
| SPI_CS0 | 11 | 7 | |
| SPI_IN | 12 | 5 | |
| PWDNB (3.3 V pull up) | 13 | 6 | 1 |
| | 14 | | |
| | 15 | | |
| | 16 | | |
| RESETB (3.3 V pull up) | 17 | 8 | 1 |
| | 18 | | |
| SPI_CS1 | 19 | 9 | |
| GND | 20 | 10 | |

In an embodiment, when the main power input, 5V, is applied to pin 2 of interface 420 of the console 130, the pin status of pins 13 and 17 of interface 420 is high, as two pins may be pulled-up to 3.3V from interface 410 of the connector element 120. By pulling-down pin 1 of interface 410 of the connector element 120 and pulling up pin 9 of interface 420, an additional low line may be achieved. In this manner, the number of potential predetermined combinations that may be used as the above-referenced “ID code” is 2³=8. As such, in this example, by reading pins 9, 13 and 17 of interface 420, compatibility between the connector element 120 and console 130 can be ensured.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

1. A system, comprising:
an array of N electrode elements configured to be attached to an external region of a patient; and
a processing device coupled to the array, the processing device configured to:
receive a set of bioelectric data signals from the array,
determine from the set of data signals a set of elements of the array that are, according to a predetermined standard, insufficiently attached to the external region, and
generate to a display device, in two dimensions, a representation of the external region and the spatial positioning of the insufficiently attached set of elements on the external region.

2. The system of claim 1, wherein the processing device is further configured to:
determine from the set of data signals a set of elements of the array that are, according to the predetermined standard, sufficiently attached to the external region, and
generate to the display device a representation of the external region and the spatial positioning of the sufficiently attached set of elements on the external region.

3. The system of claim 1, wherein the processing device is further configured to generate to the display device, in three dimensions, the representation of the external region and the spatial positioning of the insufficiently attached set of elements on the external region.

4. The system of claim 3, wherein the processing device is further configured to enable a user of the system to rotate the representation of the external region about at least one axis of the representation of the external region.

5. The system of claim 1, wherein the processing device is further configured to generate to the display device an indication of the quantity of the insufficiently attached set of elements on the external region.

6. A computer-readable medium including executable instructions that, when executed by a processing device, enable the processing device to perform a method of evaluating the quality of contact between an array of N electrode elements and an external region of a patient, the method comprising the steps of:
receiving a set of bioelectric data signals from the array;
determining from the set of data signals a set of elements of the array that are, according to a predetermined standard, insufficiently attached to the external region; and
generating to a display device, in two dimensions, a representation of the external region and the spatial positioning of the insufficiently attached set of elements on the external region.

7. The medium of claim 6, wherein the method further comprises the steps of:
determining from the set of data signals a set of elements of the array that are, according to the predetermined standard, sufficiently attached to the external region, and
generating to the display device a representation of the external region and the spatial positioning of the sufficiently attached set of elements on the external region.

8. The medium of claim 6, wherein the method further comprises the step of generating to the display device, in three dimensions, the representation of the external region and the spatial positioning of the insufficiently attached set of elements on the external region.

9. The medium of claim 8, wherein the method further comprises the step of enabling a user of the system to rotate the representation of the external region about at least one axis of the representation of the external region.

10. The medium of claim 6, wherein the method further comprises the step of generating to the display device an indication of the quantity of the insufficiently attached set of elements on the external region.

11. An electronic device, comprising:
a first electrical interface element configured to be electrically coupled to a sensor array;
a second electrical interface element configured to be electrically coupled to a processing device; and
at least one of a pull-up resistor and pull-down resistor, the at least one of a pull-up resistor and pull-down resistor enabling signal communication between the sensor array and the processing device.
